(19) United States
(12) Patent Application Publication
(10) Pub. No.: US 2016/0213673 A1
(43) Pub. Date: Jul. 28, 2016

(54) ENDURANCE FORMULATION AND USE

(71) Applicant: GLANBIA NUTRITIONALS (IRELAND) LIMITED, KILKENNY (IE)

(72) Inventors: Jeremy D. BARTOS, San Marcos, CA (US); Elaine DRUMMOND, Carlsbad, CA (US)

(21) Appl. No.: 15/003,130

(22) Filed: Jan. 21, 2016

Related U.S. Application Data
(60) Provisional application No. 62/108,207, filed on Jan. 27, 2015.

(51) Int. Cl. A61K 31/522; A61K 31/198; A23L 2/52; A61K 31/216

(52) U.S. Cl. CPC A61K 31/522; A61K 31/216; A61K 31/198; A23L 1/3051; A23L 2/52; A23L 1/302; A23L 1/304; A23L 1/30; A23V 2002/00

(57) ABSTRACT

The present disclosure provides compositions and formulations containing dihydrocapsiate, caffeine and arginine as well as methods of making and using the compositions and formulations. The compositions and formulations of the present disclosure increase human endurance during times of physical activity.

The present application claims benefit of U.S. Provisional Patent Application No. 62/108,207, filed Jan. 27, 2015, the entire contents of which is incorporated herein by reference.

The present disclosure provides compositions and formulations containing dihydrocapsiate, caffeine and arginine as well as methods of making and using the compositions and formulations. The compositions and formulations of the present disclosure increase human endurance during times of physical activity.

The compositions and formulations of the present disclosure are believed to increase or enhance endurance when ingested by sparing glycogen stores, increasing aerobic activity over anaerobic activity during times of physical activity and thereby reducing metabolite buildup that would normally lead to exhaustion and limitations on endurance by a person not ingesting a composition or formulation of the present disclosure.

Compositions and formulations of the present disclosure enhance endurance when ingested before or during, or before and during physical exertion. Increasing endurance, or the amount of time the body can work out, for example running longer distances or for longer time, increasing number of reps of workout, etc., is an increasingly important focus of sports nutrition.

A focus of the presently disclosure technology is enhancing endurance by increasing the sparing of glycogen stores, forcing aerobic activity as compared to anaerobic, increasing vasodilation, and reducing the build-up of metabolites that can lead to the feeling of exhaustion during and after physical activity.

The combination of dihydrocapsiate, caffeine and arginine provided in compositions and formulations of the present disclosure provide a unique combination designed to enhance endurance via one or more of these mechanisms.

Two of the main mechanisms for ATP synthesis are aerobic respiration (glycolysis, citric acid cycle, oxidative phosphorylation, etc.) and anaerobic respiration (glycolysis only). Aerobic respiration pathways occur in an oxygen-rich state, have a more efficient output of ATP (36-38 ATP/glucose), and are typically associated with endurance exercise. Anaerobic respiration occurs in an oxygen poor environment, has less efficient output of ATP (2 ATP/glucose), and is typically associated with intense exercise (weight lifting, sprinting, etc.). Anaerobic respiration is also much quicker than aerobic respiration (because it is only going through the glycolysis), and is only utilized by muscle cells.

In theory, if the body can endure longer periods of anaerobic respiration than aerobic respiration, or if it can utilize free fatty acids as fuel instead of glucose, it will help extend the short-term, high intensity activities associated with weight training. Once glycogen stores are used up, fatigue will also set in. Glycogen sparing is maximized with the use of non-carbohydrates as a source of energy during exercise so that the depletion of muscle glycogen is delayed. Glycogen is spared because the body burns fats for energy, making a greater contribution to an athlete's efforts during the initial stages of a race. This leaves more glycogen for the later stages of racing or exercise, for example, and muscle fatigue will be delayed.

Increasing vasodilation will also increase the rate of oxygen and glucose to the muscles, lengthening the time of aerobic respiration and further sparing glycogen stores.

Capsinoids such as dihydrocapsiate exert similar thermogenic and lipolytic effects as the red pepper compound capsaicin but without noxious sensations of pungency or “hotness.” In addition, dihydrocapsiate does not cause increases in blood pressure and heart rate like those attributed to capsaicin ingestion. The thermogenic and lipolytic effects of dihydrocapsiate (and all other capsaicins and capsinoids) are mediated through the Transient Receptor Potential Vanilloid 1 (TRPV1) receptors throughout the gastrointestinal tract, which in turn are linked with the sympathetic nervous system (SNS). When activated, they increase SNS activity which results in the downstream activation of uncoupling proteins UCP-1, UCP-2, and UCP-3.

Of particular importance for endurance is UCP-2 and UCP-3, the latter of which is expressed in the skeletal muscle of all mammals and plays a role in the transport of free fatty acids. UCP-2 is primarily expressed in adipocytes and plays a key role in lipolysis, which is the release of free fatty acids from their storage in adipocytes. This release is triggered as the body is searching for a fuel store to induce thermogenesis brought on by the TRPV-1 receptor activation (for example, in shivering).

Free fatty acids can be utilized as a fuel source in endurance training as well, and since UCP-3 is expressed in skeletal muscle and helps transport free fatty acids, it is reasonable to believe that it will help increase free fatty acid uptake by the muscles.

In one animal study (“Upregulation of uncoupling proteins by oral administration of capsiate, a nonpungent capsaicin analog” J Appl Physiol. 2003 December; 95(6):2408-15), UCP3 mRNA expression in skeletal muscle occurred within minutes of capsinoid ingestion and persisted for 2 hours. A separate animal study (“Capsiate, a Nonpungent Capsaicin Analog, Increases Endurance Swimming Capacity of Mice by Stimulation of Vanilloid Receptors” Biosci Biotechnol Biochem. 2006 April; 70(4):774-81) demonstrated that mice taking capsiate were able to swim significantly longer than mice who hadn't taken capsiate. In addition, after 30 min of swimming, the residual glycogen in the gastrocnemius muscle was higher, the serum free fatty acid concentration tended to be higher, and the serum lactic acid concentration was significantly lower in the capsiate-administered mice.

This suggests that the capsiate increased the release of free fatty acids, which UCP-3 brought to the muscle to use as fuel, sparing glycogen stores for later and thus lengthening the swimming time. This allows for increased aerobic exercise and delayed anaerobic exercise. This mechanism of action was subsequently proved in the same experiment when researchers demonstrated significantly higher oxygen consumption (aerobic) and fat oxidation (using fat as fuel) with reduced carbohydrate oxidation (glycogen sparing) following capsiate administration.

While these studies were performed using capsiate and not dihydrocapsiate, a previous study (“Assessment of the biological similarity of three capsaicin analogs (Capsinoids) found in non-pungent chili pepper (CH-19 Sweet) fruits” Biosci Biotechnol Biochem 2010; 74(2):274-8; and “Activation of transient receptor potential A1 by a non-pungent capsaicin-like compound, capsiate; Br J Pharmacol” 2012 March; 165(5):1476-86) have shown that capsiate and dihydrocapsiate have similar rates of TRVP-1 activation, suggesting that dihydrocapsiate will have a similar effect on endurance.

Respiratory quotient (RQ) is a simple measurement of the ratio of carbon dioxide eliminated to oxygen consumed. This is particularly helpful when calculating an organism's basal metabolic rate from carbon dioxide production, which is easily measured when the organism exhales. Based on the respiratory quotient one can determine if the organism's primary energy source is carbohydrates (RQ around 1) such as sugar or if it is primarily from fats (RQ around 0.7). A mixed diet of fat and carbohydrate results in an average value between 0.7 and 1 and therefore the lower the RQ the more likely the source of energy is from fats. Compounds that increase the oxidation of fats for energy, even in the presence of a carbohydrate source, can be expected to lower the RQ. In a human study (“Effects of dihydrocapsiate on adaptive and diet-induced thermogenesis with a high protein very low calorie diet: a randomized control trial” Nutrition & Metabolism 2010, 7:78), consumption of 3 mg of 9 mg of dihydrocapsiate per day for 28 days led to a significantly lower RQ than that seen in the placebo group. This was coupled with a significant increase in post prandial energy expenditure, demonstrating that dihydrocapsiate consumption increases the use of free fatty acids for ATP production, resulting in higher levels of energy available for use during endurance exercise. Caffeine has the same effect on respiratory quotient as dihydrocapsiate. A human study (Enhanced metabolic response to caffeine in exercise-trained human subjects; J Appl Physiol (1985). 1985 September; 59(3):832-7) recorded significantly lower RQ following 4 mg/kg caffeine consumption, as well as a greater increase in plasma free fatty acids, suggesting enhanced lipid oxidation following caffeine consumption.

A recent mouse study (“A single intake of capsiate improves mechanical performance and bioenergetics efficiency in contracting mouse skeletal muscle” Am J Physiol Endocrinol Metab. 2014 May 15; 306(10):E1110-9) demonstrated that although oxidative phosphorylation was not affected during rest, when exercising the mice showed increased contribution of oxidative phosphorylation to total energy turnover with a reduction in glycolysis. This further supports that glycogen sparing as oxidative phosphorylation can utilize products from fat oxidation for ATP production. In addition, ATP cost of twitch force generation was reduced while the twitch force-generating capacity was increased with the capsiate, suggesting that less energy could go farther in muscle performance while using capsiates.

Compositions and formulations of the present disclosure include caffeine as muscle glycogen sparing occurs early during endurance (aerobic) exercise following caffeine ingestion.

It is presently unclear however if glycogen sparing is due to increased fat mobilization and subsequent use by the muscle. Studies (“Caffeine and endurance performance” Sports Med. 1985 May-June; 2(3):165-74) suggest that increasing the release of adrenaline into the blood can also stimulate the release of free fatty acids from fat tissue and/or skeletal muscle. With increased fat availability to the muscle, the muscle uses less glucose early on, sparing glycogen for use later in exercise and therefore delaying fatigue.

Caffeine has been shown to decrease glycogen utilization by as much as 50% during the first 15 minutes of exercise, the result of which is saving that glycogen for later use.

Compositions and formulations of the present disclosure also include Arginine in salt forms, pure forms, conjugates with molecules such as resveratrol, and in the form of Arginine Silicate Inositol (ASI). Arginine is necessary for the synthesis of creatine in the body. Creatine is thought to build lean muscle mass and assist with short high intensity bursts (weight lifting, sprinting, etc.) Creatine has also been shown to induce glycogen sparing in animal studies. Finally, creatine is known to assist with water retention, and studies in conjunction with glycerol supplementation have demonstrated a measurable effect on hyperhydration and endurance (“The effects of creatine and glycerol hyperhydration on running economy in well trained endurance runners” J Int Soc Sports Nutr. 2011 Dec. 16; 8(1):24).

The presently disclosed compositions and formulations further increase endurance by blunting pain perception, allowing athletes to work through the warning signals the body has in place, be it exhaustion/fatigue or micro tears in the muscles that actually increase strength when healed. Caffeine has been shown to dampen pain perception through the blocking of adenosine receptors, which could result in extension of the timepoint by which the level of pain that would result in termination of exercise is reached. In addition, dihydrocapsiate may help with pain blunting through the activation of the TRPV1 receptor. Activation of TRPV1 can induce persistent depolarization of the nerve terminals, causing a decrease in their ability to generate and propagate action potentials. Together, caffeine and dihydrocapsiate are expected to blunt pain enough to increase endurance.

Compositions and formulations of the present disclosure further increase endurance by increasing vasodilation. Vasodilation is important for many different aspects of exercise. The heart has an easier time pumping blood through the circulatory system which in turn will lower the blood pressure since there is less force within the blood vessels (equal volume with greater diameter equals lower pressure). Increasing the diameter of the circulatory system will also allow the heart to pump more efficiently, and also helps to deliver key metabolic constituents such as oxygen, glucose, etc., more quickly to where they are needed. This allows for more efficient energy production, waste removal and damage repair.

When there is an adequate amount of nitric oxide in the body, the muscles around the circulatory system relax, allowing the blood vessels to widen for better blood flow, increasing the transportation rate of oxygen, nutrients, hormones, etc. to areas of need. When working out or in times of physical activity, blood flow increases to the muscles as protection and repair. In the presence of a vasodilator like nitric oxide, blood vessels open and increase blood flow. This increases both endurance and energy levels systemically. See also “Nonuniform effects of endurance exercise training on vasodilation in rat skeletal muscle” J Appl Physiol 2005 February; 98(2):753-61.

Although a known vasoconstrictor, caffeine can also help induce vasodilation by increasing intercellular calcium levels, stimulating the production of nitric oxide through the expression of endothelial nitric oxide synthase (eNOS), which can subsequently utilize free arginine to produce NO, increasing vasodilation (either increase intensity of vasodilation dose-dependently or reduce the amount of time it takes to achieve similar levels of vasodilation seen in the absence of the combination with arginine or an arginine-containing compound such as Arginine Silicate Inositol).

In endothelial cells caffeine increases intracellular calcium, stimulating the production of nitric oxide through the expression of the endothelial nitric oxide synthase enzyme. Nitric oxide (NO) is diffused to the vascular smooth muscle cells to produce vasodilation. NO is synthesized by eNOS from L-arginine and oxygen. Caffeine stimulates expression of eNOS and arginine is a precursor used by eNOS to make NO, which is in turn responsible for vasodilation. As noted above, vasodilation leads to increased blood flow to the muscles and an increase in oxygen supply, thereby reducing the environment for anaerobic respiration while also removing/reducing levels of H⁺, CO₂, and lactic acid that cause a decrease in muscle pH.

An increase of ammonia in muscle (which is a byproduct of deamination of AMP in fast-twitch fibers) leads to fatigue due to ammonotoxemia and lactic acidemia. Among other things, ammonia activates phosphofructokinase and prevents oxidation of pyruvate to acetyl CoA, thus leading to exhaustion. Both citrulline and arginine mitigate ammonia toxicity in exhausted rats by interfering with the metabolism of ammonia (“Effect of arginine, ornithine and citrulline supplementation upon performance and metabolism of trained rats” Cell Biochem Funct. 2003 March; 21(1):85-91.). Additionally, compounds that promote vasodilation will accelerate the removal of toxic byproducts of endurance exercise by increasing blood flow.

Arginase is an enzyme in the urea cycle that helps with the elimination of urea from the body. The final step in the urea cycle, arginase converts arginine and water in to ornithine and urea, the last of which is the main outlet for ammonia removal. Use of arginine in the urea pathway limits its use as a substrate for the production of creatine, nitric oxide, agmatine, glutamic acid, ornithine, proline and polyamines. However, it has also been shown that caffeine inhibits arginase activity (see “Effect of caffeine on metabolism of L-arginine in the brain” Mol Cell Biochem. 2003 February; 244(1-2):125-8). Inhibition of arginase by caffeine provides more arginine for consumption in other metabolic pathways, optimizing its use for promotion of endurance.

The combinations of dihydrocapsiate, caffeine and arginine provided in compositions and formulations of the present disclosure therefore provide a unique combination of active ingredients designed to provide enhanced endurance by multiple pathways.

U.S. Pat. No. 6,333,421 describes capsaicinoid-like substances of the following formulas:

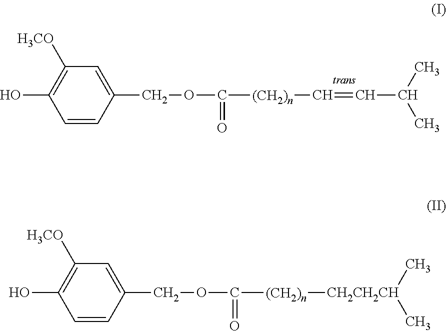

The patent describes a “sports drink” containing the capsaicinoid-like substances of the patent, orange juice concentrate, sugar, high fructose corn syrup (F-55), citric acid, salt, sodium citrate, potassium chloride, calcium phosphate, sodium glutamate, magnesium chloride, ascorbic acid, Cloudy, Emulsifying flavor, Essence and water. The patent further describes a “Chocolate” containing sugar, cocoa mass, total lipid pulverized milk, cocoa butter, lecithin, vanilla flavor, and the dried and pulverized material of “CH-19 sweet”. The patent describes the formulations of the patent as being useful for immunopotentiating activity and enhancing activity of energy metabolism

U.S. Pat. No. 7,981,460 describes substituted benzyl ester derivatives of the following formula:

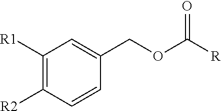

wherein R is

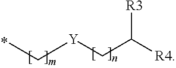

The patent describes the compounds of the patent as being useful to enhance blood circulation, in sympathetic activation action, in energy metabolism enhancing action, in immunostimulatory action, in lipolysis enhancing action, in antiobesity action, in body fat accumulation suppressive action, and in analgesic action. The patent states that the active may be used in food, pharmaceutical and cosmetic compositions.

U.S. Pat. No. 8,212,068 describes compounds of the following formula

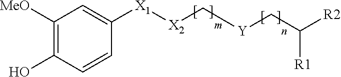

as being useful as an external blood circulation enhancer in cosmetic, pharmaceutical and food compositions.

WO 2009061051 A1 describes a capsiate- or dihydrocapsidate-containing composition and their use for preventing and treating inflammatory disease, angiogenesis-related disease and autoimmune disease or for suppressing immunity.

U.S. 20070020738 A1 describes a production method of capsinoids by dehydrating condensation, stabilizing method of capsinoid, and capsinoid composition.

U.S. 20050239883 A1 describes compositions for lowering internal lipid content with an active of the following structures:

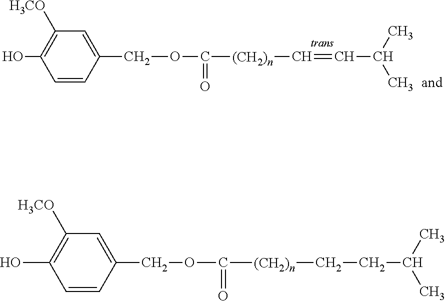

U.S. 20070141165 A1 describes method for manufacturing microcapsules containing capsinoid.

U.S. 20020068365 A1 describes a time released composition for use in the treatment of sexual disorder with compositions of L-arginine, its salts, peptides, and biological equivalents that enhances or modulates the endogenous production of nitric oxide, alone or in combination with botanical extracts for treating erectile dysfunction.

None of this art describes or suggests the combinations of dihydrocapsiate, caffeine and arginine in pure form, salt form, conjugate form, or in the form of Arginine Silicate Inositol (ASI) provided in compositions and formulations of the present disclosure.

Compositions and formulations of the present disclosure may additionally include excipients such as microcrystalline cellulose, crystalline cellulose, lactose, corn starch, sucrose, glucose; binders such as tragacanth, gum arabic, corn starch, gelatin, polyvinyl alcohol, polyvinyl ether, ethylcellulose, methylcellulose, shellac, hydroxypropylcellulose, hydroxypropyl starch, polyvinylpyrrolidone; swelling agents such as corn starch, pregelatinated starch, alginic acid, dextrin; lubricants such as magnesium stearate; flowability improving agents such as fine silicon dioxide; lubricants such as glyceryl fatty acid ester, magnesium stearate, talc, polyethylene glycol, silica, hydrogenated vegetable oil; sweetening agents such as sucrose, lactose, aspartame, acesulfame-K, sucralose, monatin, stevia, saccharin and the like; flavors to be used for various foods such as peppermint, vanilla flavor, cherry, raspberry ketone and the like; preservatives such as paraoxybenzoates, chlorobutanol, benzyl alcohol, sorbic acid; an antioxidant, such as sulfite, ascorbic acid, vitamin E, butylhydroxytoluene, sodium sulfite; and/or coating agents such as shellac, sucrose, gelatin, and hydroxypropylcellulose.

Compositions and formulations of the present disclosure may also include, for example, food additives, such as fruit juice, dextrin, cyclic oligosaccharide, cyclodextrins (alpha, beta, gamma), saccharides (monosaccharides such as fructose, glucose and/or polysaccharides), buffers, acidulant, flavor, Hiki cha powder or other taste modifiers, an emulsifier, collagen, powdered milk, polysaccharide thickener, agar or other texture modifiers, at least one vitamins, egg shell calcium, calcium pantothenate, other minerals, royal jelly, propolis, honey, dietary fibre, Agaricus subrufescens, chitin, chitosan, flavonoids, carotenoids, lutein, herbal medicine, chondroitin, and/or amino acids.

Compositions and formulations of the present disclosure may additionally or optionally include at least one of crocin, crocetin, acteoside, aloenin, aloesin, aloin, alpinetin, atractylenolide, atractylodin, aurantio-obtusin, cimigenol, cimifugin, cimiside, garcinone, ascorbic acid, astragalin, quercetin, resveratrol, pterostilbene, curcumin, demethoxycurcumin, bis-demethoxycurcumin, theaflavin, theaflavin-3,3′-digallate, theaflavin-3-gallate, theaflavin-3′-gallate, L-theanine, anthocyanidins, anthocyanins, catechin, epicatechin, gallocatechin, pepigallocatechin, epicatechin gallate, gallocatechin gallate, epigallocatechin gallate, procyanidin, chlorogenic acid, cardamonin, arctigenin, arctiin, asiatic acid, asiaticoside, bergamotin, bergapten, betaine, dioscin, galangin, cimicifugoside, cinnamic acid, ferrulic acid, fumaric acid, .alpha.-lipoic acid, carnosine, L-carnitine, caffeic acid, ellagic acid, maslinic acid, phenylethyl caffeate, caffeic acid phenethyl ester, theobromine, theophylline, caffeoylquinic acid, ursolic acid, allicin, gingerol, shogaol, ginkgolide, ginkgetin, ginsenoside, astragaloside, cycloastragenol, danshensu, danshenol, danshenxinkun, tanshinone, tanshindiol, rosmarinic acid, dosmin, nobiletin, tangeretin, luteolin, lutein, beta-lycopene, zeaxanthin, tyrosol, hyperin, hyperoside, quercetin, quercetrin, isoquercitrin, hydroxytyrosol, rosarin, β-rosasterol, rosavin, rosin, punicalagin, punicalin, myricetin, myricitrin, kaempferol, dihydromyricetin, apigenin, naringin, naringenin, honokiol, magnolol, mangiferin, mangostin, hesperetin, hesperidin, lupeol, indole-3-carbinol, genistein, genistin, daidzein, daidzin, cynarin, bilobalide, bilobetin, epimedin, sulforaphane, phloretin, phloretin-xyloglucoside, phloridzin, proanthocyanidins, procyanidin B1, procyanidin B2, procyanidin C1, silibinin, rutin, wogonin, morin, morusin, mulberroside A, mulberroside B, glycyrrhizic acid, glycyrrhetinic acid, linarin, protodioscin, protogracillin, synephrine, rebaudioside A, stevioside, vitexin, isovitexin, vitexin-4, vitexin-4″-O-glucoside, vitexin-2″-O-rhamnoside, vitexin-4′-rhamnoside, alpha-tocopherol, beta-tocopherol, gamma-tocopherol, and delta-tocopherol.

A composition or formulation of the present disclosure may additionally or optionally also include a phytochemical from at least one of the following sources: bilberry, blueberry, cranberry, raspberry, cherry, mulberry, pomegranate, maqui berry, purple corn, strawberry, grapes, black berry, gooseberry, black currants, grape, cocoa beans, coffee beans, pine bark, cardamom, cinnamon bark, ginseng, astragalus, rhodiola, garcinia, ginger, ginkgo, citrus fruit, grape skins, grape seeds, hawthorn, artichoke, broccoli, broccoli seeds, apple, olive, orange, lemon, pepper, soybean, mango, tea leaves, tomato, turmeric, cabbage, purple corn, black rice, bitter lemon, stevia, lo han, goji (wolf berry), sea buckthorn, kudzu, clove, hemp, cassia, magnolia, nutmeg, jujube, honeysuckle, poria, bellflower, lotus, basil, sesame, angelica, cimicifuga, epimedium, schisandra, salvia, licorice, ligustrum, ophiopogonis, aloe, dodder, fenugreek, gotu kola, guarana, purslane, and tribulus.

Compositions and formulations of the present disclosure may additionally and/or optionally further include—in addition to arginine in the form of ASI—one or more free amino acids or peptides, such as lysine, methionine, histidine, leucine, isoleucine, alanine, phenylalanine, asparagine, arginine, beta alanine, aspartic acid, tryptophan, proline, threonine, cysteine, selenocysteine, serine, taurine, tyrosine, valine, glycine, glutamine, glutamic acid, ornithine, carnosine, L-carnitine, glutathione.

Compositions and formulations of the present disclosure may additionally and/or optionally include one or more vitamins or minerals, including, for example, vitamin A, vitamin C, vitamin B1, vitamin B2, vitamin B3, vitamin B5, vitamin B6, vitamin B12, vitamin D, vitamin E, vitamin K (and/or derivatives), folic acid, biotin, calcium, sodium, potassium, phosphorus, chromium, vanadium, magnesium, zinc, manganese, selenium, copper, molybdenum, boron, vanadium, and/or iron (and/or derivatives).

Compositions and formulations of the present disclosure may also include creatine and derivatives thereof, capsiate, carnitine, nordihydrocapsiate, pterostilbene and other polyphenols, and agmatine (or agmatine sulfate).

Compositions and formulations of the present disclosure may further optionally include citrus bioflavonoids (such as quercetin, rutin, isoquercetin, synephrine, and octopamine); CoQ10, thiamine, citrulline malate, nicotinamide adenoside dinucleotide (NAD), nicotinamide riboside (NR), citrulline, lutein, lycopene, capsaicin, arginine alpha ketoglutarate (Arginine AKG), L-arginine pyroglutamate, arginine ketoisocaproate, citric acid, ornithine alpha ketoglutarate (Ornithine AKG), omega-3 fatty acids (DHA and EPA), L-norvaline, nitrate, taurine, arginine ethyl ester, carnosine, vanadyl sulfate, L-alpha glycerophosphorylcholine (Alpha GPC), Pinus pinaster (Pycnogenol®), turmeric (curcumin, demethoxycurcumin, bis-demethoxycurcumin), rutacaerpine, Epimedium spp., garlic (allicin, alliin, and the like), flaxseed, flaxseed ligans (alpha linolenic acid (ALA), gamma linolenic acid (GLA)), Schisandria (such as Schisandrin, Schisandrol A and B, and Gamma schisandrin), green tea catechins (such as catechin, ECGC, ECG, and EGC), black tea, dong quai (ligustilide), Andrographis (Andrographolides), grape extract (such as resveratrol), anthocyanins (such as cyanidin 3-glucoside (C3G), cyanidin 3-rutinoside, delphinidin 3-glucoside, and malvidin 3-glucoside), Danshen, Beta vulgaris root, celery (3-N-butylphthalide), Berberine, Feverfew, Jasmine, Lemon balm, vinpocetine, Lotus, White horehound, Lemongrass, Yerba mate, Peony, Mustard, Motherwort, Cramp bark, Grapeseed, Proanthocyanidins (PACs, including Procyanidin A1, Procyanidin A2, Procyanidin B1, Procyanidin B2, and the like), Spinach (containing nitrates), Kale (containing nitrates), Broccoli (containing nitrates), Beet (containing nitrates), theobromine, theophylline, phenylethylamine (PEA), and the like), Hawthorn, Hawthorn flavonoids (hyperoside, vitexin, isovitexin, and the like), Catauba extract, apple polyphenols, and combinations thereof.

Compositions and formulations of the present disclosure may further also include NO-inducers such as glyceryl trinitrate (a.k.a. nitroglycerin), isosorbide mononitrate, isosorbide dinitrate, clonitrate, ettriol trinitrate (ETTN), erythrityl tetranitrate, pentaerythritol tetranitrate (PETN), pentrinitrol, D-mannitol hexanitrate, trolnitrate phosphate, sodium nitroprusside, PDE5 inhibitors (slidenafil, tadalafil, vardenafil), papaverine, bamethan, bencyclane, beraprost, betahistine, brovincamine, bufeniode, buflomedil, butalamine, cetiedil, chromonar, ciclonicate, cinepazide, cinnarizine, clobenfurol, cloricromen, cyclandelate, dilazep, droprenilamine, eburnamonine, efloxate, eledoisin, etafenone, fasudil, fendiline, fenoxedil, flunarizine, hexobendine, ibudilast, ifenprodil, iloprost, inositol niacinate, itramin tosylate, kallidin, kallikrein, khellin, lidoflazine, lomerizine, moxisylyte, nafronyl, nicotinyl alcohol, nimodipine, nylidrin, pentifylline, pimefylline, piribedil, trapidil, trimetazidine, vincamine, vinpocetine, viquidil, visnadine, xanthinol niacinate, bendazol, floredil, medibazine, tinofedrine, amotriphene, benfurodil hemisuccinate, hepronicate, hepronicate, nicofuranose, suloctidil, or salts and/or prodrugs thereof.

Compositions and formulations of the present disclosure may additionally or optionally contain, for example, carrier materials such as corn starch, acacia, gelatin, malt, tragacanth, microcrystalline cellulose, kaolin, dicalcium phosphate, calcium carbonate, sodium chloride, or alginic acid; disintegrators including, microcrystalline cellulose, or alginic acid; binders including acacia, methylcellulose, ethyl cellulose, sodium carboxymethylcellulose, polyvinylpyrrolidone, or hydroxypropyl methylcellulose; and lubricants such as magnesium stearates, stearic acid, silicone fluid, talc, oils, waxes, or colloidal silica. Compositions and formulations of the present disclosure may additionally include mixtures of acids (like citric, tartaric, malic and fumaric acid or combination thereof) and carbonates like sodium, potassium bicarbonate or carbonate; water soluble binders (starches, natural gums, cellulose gums, microcrystalline cellulose, methylcellulose, cellulose ethers, ethylcellulose, sodium carboxymethylcellulose, gelatin, dextrose, lactose, sucrose, sorbitol, mannitol, polyethylene glycol, polyvinylpyrrolidone, pectins, alginates, polyacrylamides, polyvinyloxoazolidone, polyvinylalcohols and mixtures thereof) and/or lubricants (sodium benzoate, polyethylene glycol, L-leucine, adipic acid, and combinations thereof). A composition or formulation of the present disclosure may also optionally include other ingredients including, e.g., flavor agents, fillers, surfactants, color agents, and sweeteners.

Compositions and formulations of the present disclosure may additionally include, for example, water or other aqueous formulations including suspending agents such as, for example, alginates, pectin, kelgin, carrageenan, acacia, methylcellulose, polyvinyl alcohol, or polyvinylpyrrolidone. The compositions or formulations of the present disclosure may be in the form of, for example, a solution (aqueous or nonaqueous [not containing added water]), emulsion, syrup, gel, powder or elixir including or containing wetting agents, sweeteners, and coloring and flavoring agents. Various liquid and powder compositions and formulations according to the present disclosure may be prepared by conventional methods.

Compositions and formulations of the present disclosure may include aqueous or nonaqueous carriers, diluents, fillers, binders, humectants, disintegrating agents, solution retarders, absorption accelerators, wetting agents, absorbents, or lubricating agents, solvents or vehicles including water ethanol, polyols (propylene glycol, polyethylene glycol, or glycerol), suitable mixtures thereof, vegetable oils (such as olive oil) and organic esters such as ethyl oleate. Further, the compositions and formulations of the present disclosure may include stabilizers, solubilizers, suspending agents, emulsifiers, soothing agents, buffers, preservatives, isotonic agents, or antibacterial and antifungal agents. Other useful components may include magnesium stearate, calcium stearate, mannitol, xylitol, erythritol, maltodextrin, sweeteners, starch, carboxymethylcellulose, microcrystalline cellulose, silica, gelatin, and silicon dioxide.

Compositions and formulations of the present disclosure may optionally and/or additionally be formulated for administration, and be administered, for example, orally, parenterally (intravenous, intramuscular, intraperitoneal, intrasternally, subcutaneously, intraarticular injection and infusion), or by percutaneous, rectal, mucosal, intranasal or topical (transdermal, as by powders, ointments, creams, sprays, drops or patches) administration.

Compositions and formulations of the present disclosure optionally contain free arginine.

Compositions and formulations of the present disclosure optionally contain arginine in a salt form, such as arginine HCl.

Compositions and formulations of the present disclosure optionally contain arginine only in the form of Arginine Silicate Inositol (ASI).

Compositions and formulations of the present disclosure optionally contain arginine only in conjugate with resveratrol.

Compositions and formulations of the present disclosure optionally do not include cacao or cacao mass or theobromine (3,7-dimethylxanthine or 3,7-dimethyl-1H-purine-2,6-dione).

Compositions and formulations of the present disclosure optionally do not include capsaicin or capsaicinoids. Compositions and formulations of the present disclosure optionally do not include capsaicin, dihydrocapsaicin, nordihydrocapsaicin, homocapsaicin, and/or homodihydrocapsaicin, while optionally including dihydrocapsiate. Compositions and formulations of the present disclosure optionally do not include N-(4-hydroxy-3-methoxybenzyl)-8-methylnon-6-enamide or trans-8-Methyl-N-vanillylnon-6-enamide (CAS Registry Number 404-86-4) or N-[(4-Hydroxy-3-methoxy-phenyl) methyl]-8-methyl-nonanamide (CAS Registry Number 19408-84-5).

Compositions and formulations of the present disclosure optionally do not include allyl isothiocyanate (and/or optionally mustard, horseradish or wasabi containing same), or piperine (and/or black pepper or white pepper containing the same), or ginger (and/or any one or a mixture of zingerone, shogaol, and gingerol).

Compositions of the present disclosure may be formulated as comestibles—including fruit-based drinks, coffee-based drinks, tea-based drinks, sport drinks, nutrition bars, snack foods, gums, cereals, candies, energy drinks, adult nutritional drinks, health drinks, and other food products. Sports drinks, energy drink and adult nutritional drinks described herein include, for example, beverages that rehydrate athletes, as well as restoring electrolytes, carbohydrates and other nutrients, beverages that include legal stimulants, electrolytes, vitamins and minerals, and beverages that provide nutritional support. Such sports drinks, sports products, bars, energy drinks and adult nutritional drinks include, for example, Gatorade, POWERade, and All Sport, BSN Amino X, Scivation XTend Endurance, Twin Lab Endurance Fuel, Power Bar Power Gel, Gu Chomps Energy Chews, Clif Bar Energy Bar, Stinger Energy Bar, Power Bar Energy Bar, Power Bar Endurance Drink, Monster Energy Drink, 5 Hour Energy, Jolt Cola, RockStar, NOS Energy Drink, Red Bull, Ensure and Longetics. Health drinks include beverages that have a beneficial health effect, such as reducing inflammation, supporting the immune system, neutralizing infectious agents, preventing blocked arteries, preserving cognitive function and inhibiting cancer growth.

Compositions of the present disclosure may also be drinks or formulations that are not coffee-based drinks or are not coffee-based formulations, or are not tea-based drinks, or are not tea-based formulations.

Compositions and formulations of the present disclosure may be administered to a mammal in need of increased and/or enhanced endurance.

An individual subject benefiting from the compositions and formulations of the present disclosure may be an animal, mammal or a human. Animal or mammal subjects include large domestic mammals, for example, cattle (or other bovine species), horses, pigs, sheep, goats, and other livestock. Animal or mammal subjects may also include smaller domestic mammals, such as, but not limited to, dogs, cats, rabbits, and rodents including rats, mice, hamsters, gerbils, and guinea pigs.

Compositions and formulations of the present disclosure may be administered in a manner and at intervals to increase endurance. The compositions and formulations of the present disclosure may be administered, for example, prior to and/or during and/or after physical activity, such as running, playing sports, weight lifting, etc.

For example, as a preworkout supplement the combination may be part of a ready to mix drink powder that is added to 10-12 ounces of cold water and consumed 30 minutes prior to the beginning of a workout.

The present disclosure provides compositions containing dihydrocapsiate, caffeine and various forms of arginine. Compositions of the present disclosure alternatively or optionally do not contain capsaicin or a capsaicinoid. Compositions of the present disclosure alternatively or optionally do not contain at least one of high fructose corn syrup, sucrose, maltose or lactose.

Compositions of the present disclosure may be in the form of a unit dosage or serving for a person. Compositions of the present disclosure may be in the form of a ready-to-mix (RTM) or ready-to-drink (RTD) formulation.

Compositions of the present disclosure may be in the form of a gel, chew, powder, capsule, tablet, satchel, bar or drink. Compositions of the present disclosure may be in the form of a fruit drink, a coffee, a tea, an energy drink, an adult nutritional drink, an inhalant, a health drink, a lozenge, a supplement, a tablet, a capsule or a sports drink.

Compositions of the present disclosure may contain dihydrocapsiate in an amount in the range of 10 μg to 50 mg, such as in the range of 20 μg to 50 mg, or in the range of 30 μg to 50 mg, or in the range of 40 μg to 50 mg, or in the range of 50 μg to 50 mg, or in the range of 60 μg to 50 mg, or in the range of 70 μg to 50 mg, or in the range of 80 μg to 50 mg, or in the range of 90 μg to 50 mg, or in the range of 10 μg to 40 mg, or in the range of 10 μg to 30 mg, or in the range of 10 μg to 20 mg, or in the range of 10 μg to 10 mg, or in the range of 10 μg to 5 mg, or in the range of 10 μg to 10 mg, or in the range or 100μ to 5 mg, or in the range of 100 μg to 3 mg, or in the range of 3 mg to 5 mg, or in ranges intermediate of any one of these ranges.

Compositions of the present disclosure may contain capsaicin in addition to or in place of dihydrocapsiate in an amount in the range of 10 μg to 50 mg, such as in the range of 20 μg to 50 mg, or in the range of 30 μg to 50 mg, or in the range of 40 μg to 50 mg, or in the range of 50 μg to 50 mg, or in the range of 60 μg to 50 mg, or in the range of 70 μg to 50 mg, or in the range of 80 μg to 50 mg, or in the range of 90 μg to 50 mg, or in the range of 10 μg to 40 mg, or in the range of 10 μg to 30 mg, or in the range of 10 μg to 20 mg, or in the range of 10 μg to 10 mg, or in the range of 10 μg to 5 mg, or in the range of 10 μg to 10 mg, or in ranges intermediate of any one of these ranges.

Composition of the present disclosure may contain arginine in an amount in the range of 10 mg to 15 g, such as in the range of 60 mg to 15 g, or in the range of 70 mg to 15 g, or in the range of 90 mg to 15 g, or in the range of 90 mg to 15 g, or in the range of 100 mg to 15 g, or in the range of 10 mg to 10 g, or in the range of 10 mg to 5 g, or in the range of 10 mg to 1 g, or in ranges intermediate of any one of these ranges.

Compositions of the present disclosure may contain ASI in an amount in the range of 10 mg to 10 g, or in the range of 20 mg to 10 g, or in the range of 30 mg to 10 g, or in the range of 40 mg to 10 g, or in the range of 50 mg to 10 g, or in the range of 60 mg to 10 g, or in the range of 70 mg to 10 g, or in the range of 80 mg to 10 g, or in the range of 90 mg to 10 g, or in the range of 100 mg to 10 g, or in the range of 10 mg to 9 g, or in the range of 10 mg to 8 g, or in the range of 10 mg to 7 g, or in the range of 10 mg to 6 g, or in the range of 10 mg to 5 g, or in the range of 10 mg to 4 g, or in the range of 10 mg to 3 g, or in the range of 10 mg to 2 g, or in the range of 10 mg to 1 g, or in ranges intermediate of any one of these ranges.

Compositions of the present disclosure may contain caffeine in an amount in the range of 2 mg to 400 mg, or in the range of 3 mg to 400 mg, or in the range of 4 mg to 400 mg, or in the range of 5 mg to 400 mg, or in the range of 10 mg to 400 mg, or in the range of 20 mg to 400 mg, or in the range of 30 mg to 400 mg, or in the range of 40 mg to 400 mg, or in the range of 50 mg to 400 mg, or in the range of 60 mg to 400 mg, or in the range of 70 mg to 400 mg, or in the range of 80 mg to 400 mg, or in the range of 90 mg to 400 mg, or in the range of 100 mg to 400 mg, or in the range of 2 mg to 350 mg, or in the range of 2 mg to 300 mg, or in the range of 2 mg to 250 mg, or in the range of 2 mg to 200 mg, or in the range of 2 mg to 150 mg, or in the range of 2 mg to 100 mg, or in the range of 2 mg to 50 mg, or in the range of 2 mg to 40 mg, or in the range of 2 mg to 25 mg, or in the range of 2 mg to 10 mg, or in the range of 2 mg to 5 mg, or in ranges intermediate of any one of these ranges.

Compositions of the present disclosure may contain a ratio, by weight, of caffeine to dihydrocapsiate in the range of 40000:1 to 8:1, or in the range of 40000:1 to 8:1, or in the range of 30000:1 to 8:1, or in the range of 20000:1 to 8:1, or in the range of 10000:1 to 8:1, or in the range of 5000:1 to 8:1, or in the range of 2500:1 to 8:1, or in the range of 1000:1 to 8:1, or in the range of 500:1 to 8:1, or in the range of 100:1 to 8:1, or in the range of 50:1 to 8:1, or in the range of 40000:1 to 25:1, or in the range of 40000:1 to 25:1, or in the range of 30000:1 to 25:1, or in the range of 20000:1 to 25:1, or in the range of 10000:1 to 25:1, or in the range of 5000:1 to 25:1, or in the range of 2500:1 to 25:1, or in the range of 1000:1 to 25:1, or in the range of 500:1 to 25:1, or in the range of 250:1 to 25:1, or in the range of 100:1 to 25:1, or in the range of 50:1 to 25:1, or in the range of 25:1 to 8:1, or in ranges intermediate of any one of these ranges.

Compositions of the present disclosure may contain a ratio, by weight, of arginine to caffeine in the range of 1:10 to 100:1, or in the range of 2:10 to 100:1, or in the range of 5:10 to 100:1, or in the range of 1:1 to 100:1, or in the range of 2:1 to 100:1, or in the range of 3:1 to 100:1, or in the range of 4:1 to 100:1, or in the range of 5:1 to 100:1, or in the range of 10:1 to 100:1, or in the range of 20:1 to 100:1, or in the range of 30:1 to 100:1, or in the range of 40:1 to 100:1, or in the range of 50:1 to 100:1, or in the range of 60:1 to 100:1, or in the range of 70:1 to 100:1, or in the range of 80:1 to 100:1, or in the range of 90:1 to 100:1, or in ranges intermediate of any one of these ranges.

The present disclosure provides a method of enhancing endurance in a mammal, such as a human (and alternatively a horse, dog, mouse, hamster, rat, etc.), by administering a composition of the present disclosure to the mammal in a sufficient amount and over a course of time so as to enhance the endurance of the mammal, the enhanced endurance for example being measured by an increase in the maximum running time of the mammal and/or maximum swimming time of the mammal and/or other physical activity as compared to the maximum running time, swimming time and/or other physical activity of the mammal prior to being administered the composition.

The present disclosure provides a method of enhancing the endurance of a mammal, such as a human (and alternatively a horse, dog, mouse, hamster, rat, etc.), by administering a composition of the present disclosure to the mammal in a sufficient amount and over a course of time so as to enhance the endurance of the mammal, the enhanced endurance for example being measured by an increase in the maximum repetitions of physical activity, such as in weight lifting, as compared to the maximum repetitions of the mammal prior to being administered the composition.

The present disclosure provides a method of enhancing the endurance of a mammal, such as a human (and alternatively a horse, dog, mouse, hamster, rat, etc.), by administering a composition of the present disclosure to the mammal in a sufficient amount and over a course of time so as to enhance the endurance of the mammal, the enhanced endurance being measured for example by a decrease in the rating of perceived exertion (RPE) during physical exertion, such as during running, swimming, weight lifting and/or walking, as compared to the RPE of the mammal prior to being administered the composition.

The present disclosure provides a method of enhancing the respiratory quotient (RQ)—by decreasing the respiratory quotient—in a mammal, such as a human (and alternatively a horse, dog, mouse, hamster, rat, etc.), by administering a composition of the present disclosure to the mammal in a sufficient amount and over a course of time so as to enhance the respiratory quotient of the mammal. Enhancing the respiratory quotient of a mammal according to the present disclosure results in enhancing the availability of free fatty acids to the mammal, and reducing the respiratory quotient, as a result of administering a composition of the present disclosure to the mammal. The present disclosure provides a method of increasing the levels of free fatty acids in a mammal, leading to greater availability of the fatty acids for use in ATP production and ultimately increasing endurance for physical activity, such as, for example, weightlifting, biking, running, swimming.

Although not limited, the course of time of administration of a composition of the present disclosure in a method of enhancing the endurance of a mammal according to the present disclosure may be in the range of 1 day to 4 weeks, 2 days to 4 weeks, or in the range of 3 days to 4 weeks, or in the range of 4 days to 4 weeks, or in the range of 5 days to 4 weeks, or in the range of 6 days to 4 weeks, or in the range of 7 days to 4 weeks, or in the range of 1 days to 27 days, or in the range of 1 days to 26 days, or in the range of 1 day to 25 days, or in the range of 1 day to 24 days, or in the range of 1 day to 23 days, or in the range of 1 day to 22 days, or in the range of 1 day to 21 days, or in the range of 1 day to 20 days, or in the range of 1 day to 19 days, or in the range of 1 day to 18 days, or in the range of 1 day to 17 days, or in the range of 1 day to 16 days, or in the range of 1 day to 15 days, or in the range of 1 day to 14 days, or in ranges intermediate of any one of these ranges.

The present disclosure provides a method of enhancing the endurance of a mammal, such as a human (and alternatively a horse, dog, mouse, hamster, rat, etc.), by 5 to 20%, or in the range of 5 to 20%, or in the range of 6 to 20%, or in the range of 7 to 20%, or in the range of 8 to 20%, or in the range of 9 to 20%, or in the range of 10 to 20%, or in the range of 11 to 20%, or in the range of 12 to 20%, or in the range of 13 to 20%, or in the range of 14 to 20%, or in the range of 15 to 20%, or in the range of 16 to 20%, or in the range of 17 to 20%, or in the range of 18 to 20%, or in the range of 19 to 20%, or in the range of 1 to 19%, or in the range of 1 to 18%, or in the range of 1 to 17%, or in the range of 1 to 16%, or in the range of 1 to 15%, or in the range of 1 to 14%, or in the range of 1 to 13%, or in the range of 1 to 12%, or in the range of 1 to 11%, or in the range of 1 to 10%, or in the range of 1 to 9%, or in the range of 1 to 8%, or in the range of 1 to 7%, or in the range of 1 to 6%, or in the range of 1 to 5%, or in the range of 1 o 4%, or in the range of 1 to 3%, or in the range of 1 to 2%, or in ranges intermediate of any one of these ranges.

The present disclosure provides a method of enhancing the endurance of a mammal, such as a human (and alternatively a horse, dog, mouse, hamster, rat, etc.), wherein the rating of perceived exertion is decreased by at least 1, or at least 2, or at least 3 or at least 4 on a scale of 1 to 10 as a measure of enhanced endurance.

The presently disclosed technology is further illustrated by the following non-limiting examples of compositions and formulations. Note that examples only include active ingredients and do not include optional sweeteners, flavors, colors, excipients, and the like.

### EXAMPLE 1

### Ready to Mix Beverage Powder for Preworkout

Caffeine: 150 mg

Dihydrocapsiate: 2 mg

Arginine Base: 3500 mg

Leucine: 1500 mg

Isoleucine: 750 mg

Valine: 750 mg

Glutamine: 1000 mg

Beta-Alanine: 500 mg

Quercetin: 50 mg

Betaine: 100 mg

Glycerol: 1000 mg

### EXAMPLE 2

### Protein Bar

Caffeine: 5 mg

Dihydrocapsiate: 3 mg

Arginine Alpha Keto Glutarate (AAKG): 900 mg

Whey Protein Concentrate: 12 grams

Whey Protein Isolate: 6 grams

Hydrolyzed Whey Protein: 2 grams

Pterostilbene: 25 mg

Vitamin B5: 1 mg

Vitamin B12: 2 mcg

Vitamin C: 10 mg

Vitamin E: 5 IU

Magnesium: 10 mg

Chromium: 20 mcg

### EXAMPLE 3

### Ready to Drink Energy Beverage

Caffeine: 75 mg

Dihydrocapsiate: 1 mg

Arginine Silicate Inositol Complex: 300 mg

Panax ginseng: 45 mg

Taurine: 400 mg

Ginkgo biloba: 15 mg

Citric Acid: 50 mg

### EXAMPLE 4

### Sports Chew

Caffeine: 5 mg

Dihydrocapsiate: 100 mcg

Arginine Silicate Inositol Complex: 100 mg

L-Carnitine: 50 mg

Choline: 10 mg

N-Acetyl Cysteine: 50 mg

Resveratrol: 30 mg

Potassium: 35 mg

Sodium: 48 mg

Magnesium: 20 mg

### EXAMPLE 5

A study was conducted to assess the combinatory effect of dihydrocapsiate, arginine and caffeine (DAC) in an exercising population. The study group consisted of 10 healthy, recreationally fit males, aged 27±2 years, with an average BMI of 26.9±1.4 kg/m². The study was designed in a single blind, placebo-controlled, crossover fashion so that each of the 10 males completed both the treatment and placebo arm. The treatment consisted of a ready to mix (RTM) beverage containing 3 mg dihydrocapsiate, 100 mg arginine and 40 mg caffeine: the placebo was a calorie and flavor-matched RTM lacking any bioactives. Indirect calorimetry was utilized to measure resting metabolic rate (RMR) rate prior to baseline blood and vital sign measurements. Each subject consumed either the treatment or placebo 30 minutes before engaging in a series of eccentric exercises which consisted of 4 sets of repetitions to failure of bench press at 100% of body weight and leg extension at 30% of body weight. The subjects' maximum repetitions to failure (MRF) performance and rate of perceived exhaustion (RPE) were recorded. At time points 60, 120 and 180 minutes post-treatment and exercise, the RMR, vital signs and blood sample measurements were repeated.

At 60 and 120 minutes post-treatment and exercise, oxygen consumption (expressed in absolute units as well as relative to body weight) was greater in response to DAC treatment than placebo. Similarly, respiratory quotient (RQ) was lower at 120 and 180 minutes post-treatment and exercise after consumption of DAC treatment. The lowering of the RQ is a result of the depletion of stored lipids. These data were reflected in the serum and plasma markers of lipolysis in that glycerol and free fatty acids tended to be higher in response to DAC treatment over the entire post-exercise period; with fatty acids increased by 35.5% above placebo and glycerol increased by 2.13%.

There was an overall trend for increased maximal repetitions to failure for bench press and leg extension in response to treatment, with movements increasing by 2.9% and 1.6%, respectively. This coincided with a lower heart rate after exercise in response to treatment (65 bpm) versus placebo (67 bpm), which would indicate a greater sense of comfort in performing max effort movements. For example, one 27 year old male improved his maximal repetitions to failure for bench press by 11.8% and leg extension by 14.3%, with a simultaneous drop in respiratory quotient of 15.3% and an increase in circulating free fatty acids of 50% compared with his performance after placebo. This individual's heart rate remained stable across all measures. Looking at the top performing tertile in the population, after consuming the treatment the average improved bench press was 10.1%, leg extension was 21.7%, respiratory quotient dropped by 0.6% and free fatty acids increased by 30% compared with placebo.

While in the foregoing specification this presently disclosed technology has been described in relation to certain embodiments thereof, and many details have been put forth for the purpose of illustration, it will be apparent to those skilled in the art that the presently disclosed technology is susceptible to additional embodiments and that certain of the details described herein can be varied considerably without departing from the basic principles of the disclosed technology.

The use of the terms “a,” “an,” “the,” and similar referents in the context of describing the presently disclosed technology will be understood to include both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the presently disclosed technology and does not pose a limitation on the scope of the technology unless otherwise recited.

All references cited herein are incorporated by reference in their entirety. The presently disclosed technology may be embodied in other specific forms without departing from the spirit or essential attributes thereof.

1. A composition comprising dihydrocapsiate, caffeine and arginine.

2. The composition of claim 1 wherein said composition does not contain capsaicin or other capsaicinoid.

3. The composition of claim 1 wherein said composition does not contain at least one of high fructose corn syrup, sucrose, maltose or lactose.

4. (canceled)

5. The composition of claim 1 in the form of a ready-to-mix (RTM) or ready-to-drink (RTD) formulation.

6. The composition of claim 1 in the form of a gel, chew, bar or drink.

7. The composition of claim 1 in the form of a fruit drink, a coffee, a tea, an energy drink, an adult nutritional drink, an inhalant, a health drink, a lozenge, a supplement, a tablet, a capsule or a sports drink.

8. The composition of claim 1 wherein the dihydrocapsiate is present in the formulation in an amount in the range of 10 μg to 50 mg.

9. (canceled)

10. The composition of claim 1 wherein the arginine is present in the formulation in an amount in the range of 10 mg to 15 g.

11. (canceled)

12. The composition of claim 1 wherein the ASI is present in the formulation in place of or in addition to arginine in an amount in the range of 10 mg to 10 g.

13. (canceled)

14. The composition of claim 1 wherein the caffeine is present in the formulation in an amount in the range of 2 mg to 400 mg.

15. (canceled)

16. The composition of claim 8 wherein the arginine is present in the formulation in an amount in the range of 10 mg to 15 g.

17. (canceled)

18. The composition of claim 8 wherein the ASI is present in the formulation in place of or in addition to arginine in an amount in the range of 10 mg to 10 g.

19. The composition of claim 8 wherein the ASI is present in the formulation in place of or in addition to arginine in an amount in the range of 10 mg to 10 g and wherein capsaicin is present in the formulation in place of or in addition to dihydrocapsiate in an amount in the range of 10 μg to 50 mg.

20. (canceled)

21. The composition of claim 8 wherein the caffeine is present in the formulation in an amount in the range of 2 mg to 400 mg.

22. (canceled)

23. The composition of claim 10 wherein the ASI is present in the formulation in place of or in addition to arginine in an amount in the range of 10 mg to 10 g.

24. (canceled)

25. The composition of claim 10 wherein the caffeine is present in the formulation in an amount in the range of 2 mg to 400 mg.

26. (canceled)

27. The composition of claim 12 wherein the caffeine is present in the formulation in an amount in the range of 2 mg to 400 mg.

28. (canceled)

29. The composition of claim 1 wherein the dihydrocapsiate is present in the formulation in an amount in the range of 10 μg to 50 mg, the arginine is present in the formulation in an amount in the range of 10 mg to 15 g and the caffeine is present in the formulation in an amount in the range of 2 mg to 400 mg.

30. (canceled)

31. The composition of claim 1 wherein the dihydrocapsiate is present in the formulation in an amount in the range of 10 μg to 50 mg, the arginine content in the formulation in an amount in the range of 10 mg to 20 g, and the caffeine is present in the formulation in an amount in the range of 2 mg to 400 mg.

32. (canceled)

33. The composition of claim 1 wherein the dihydrocapsiate is present in the formulation in an amount in the range of 10 μg to 50 mg, the arginine is present in the formulation in an amount in the range of 50 mg to 15 g, the ASI is present in the formulation in an amount in the range of 10 mg to 10 g, and the caffeine is present in the formulation in an amount in the range of 2 mg to 400 mg.

34. (canceled)

35. The composition of claim 1 wherein the ratio, by weight, of caffeine to dihydrocapsiate is in the range of 40000:1 to 8:1.

36. The composition of claim 1 wherein the ratio, by weight, of caffeine to dihydrocapsiate is in the range of 250:1 to 25:1.

37. The composition of claim 1 wherein the ratio, by weight, of arginine to caffeine is in the range of 1:10 to 100:1.

38. The composition of claim 1 wherein the ratio, by weight, of arginine to caffeine is in the range of 1:2 to 30:1.

39. A method of enhancing endurance in a mammal comprising administering a composition of claim 1 to said mammal in a sufficient amount and over a course of time so as to enhance the endurance of said mammal, said enhanced endurance being measured by an increase in the maximum running time of said mammal and/or maximum swimming time of said mammal and/or other physical activity as compared to the maximum running time, swimming time and/or other physical activity of said mammal prior to being administered the composition.

40. A method of enhancing the endurance of a mammal comprising administering a composition of claim 1 to said mammal in a sufficient amount and over a course of time so as to enhance the endurance of said mammal, said enhanced endurance being measured by an increase in the maximum repetitions of physical activity, such as in weight lifting, as compared to the maximum repetitions of said mammal prior to being administered the composition.

41. A method of enhancing the endurance of a mammal comprising administering a composition of claim 1 to said mammal in a sufficient amount and over a course of time so as to enhance the endurance of said mammal, said enhanced endurance being measured by a decrease in the rating of perceived exertion (RPE) during physical exertion, such as during running, swimming, weight lifting and/or walking, as compared to the RPE of said mammal prior to being administered the composition.

42. The method of claim 39 wherein said mammal is a human.

43. The method of claim 39 wherein said course of time is 1 day to 12 weeks.

44. The method of claim 39 wherein the endurance is increased by 5 to 20%.

45. The method of claim 41 wherein the rating of perceived exertion is decreased by at least 1 on a scale of 1 to 10.

46. A method of enhancing the respiratory quotient of a mammal comprising administering a composition of claim 1 to said mammal in a sufficient amount and over a course of time so as to enhance the availability of free fatty acids to said mammal.
